(19) United States
(12) Patent Application Publication
(10) Pub. No.: US 2024/0198011 A1
(43) Pub. Date: Jun. 20, 2024

(54) Electronic Encoder Module and Drug Delivery Device Herewith

(71) Applicant: Sanofi, PARIS (FR)

(72) Inventors: Alexander Allerdings, Frankfurt am Main (DE); Jonathan Jason Drake, Warwick, Warwickshire (GB); Tom Alexander Earwaker, Warwick, Warwickshire (GB); Matthew Meredith Jones, Warwick, Warwickshire (GB); Aidan Michael O`Hare, Warwick, Warwickshire (GB)

(21) Appl. No.: 18/287,797

(22) PCT Filed: Apr. 22, 2022
(86) PCT No.: PCT/EP2022/060642

§ 371 Date: Oct. 20, 2023

(30) Foreign Application Priority Data
Apr. 23, 2021 (EP) 21315069.1
Jan. 10, 2022 (EP) 22315005.3

(51) Int. Cl. A61M 5/315

(52) U.S. Cl. CPC A61M 5/31568; A61M 5/31546; A61M 2205/52; A61M 2205/8237

(57) ABSTRACT

The present invention refers to an electronic encoder module (100) for use with a drug delivery device (1) for recording doses that are delivered from the drug delivery device (1) or with a charging station (200). The electronic module comprises a rechargeable electrical power supply (103), a memory, a processor with a PCBA (102) configured to control operation of the electronic module and coupled to the electrical power supply (103) and to the memory, at least one sensor arrangement (104) and a coupling portion for releasably mounting the electronic module (100) to the drug delivery device (1) when it is not mounted to a charging station (200) or to the charging station (200) when it is not mounted to the drug delivery device (1). At least one electrical power transfer component (107) is located within the coupling portion and connected to the PCBA (102) and/or to the rechargeable electrical power supply (103).

The present invention is generally directed to an electronic encoer module for a drug delivery device and to a dose recording system comprising a drug delivery device and an electronic module to be attached to the drug delivery device.

Pen type drug delivery devices have application where regular injection by persons without formal medical training occurs. This may be increasingly common among patients having diabetes where self-treatment enables such patients to conduct effective management of their disease. In practice, such a drug delivery device allows a user to individually select and dispense a number of user variable doses of a medicament.

There are basically two types of drug delivery devices: resettable devices (i.e., reusable) and non-resettable (i.e., disposable). For example, disposable pen delivery devices are supplied as self-contained devices. Such self-contained devices do not have removable pre-filled cartridges.

Rather, the pre-filled cartridges may not be removed and replaced from these devices without destroying the device itself. Consequently, such disposable devices need not have a resettable dose setting mechanism. The present invention is especially applicable for disposable devices although use in reusable devices would be possible, too.

For such devices the functionality of recording doses that are dialed and delivered from the pen may be of value to a wide variety of device users as a memory aid or to support detailed logging of dose history. Thus, drug delivery devices using electronics are becoming increasingly popular in the pharmaceutical industry as well as for users or patients. For example, a dose recording system is known from WO 2021/116387 A1 comprising a drug delivery device and an electronic module which is removably mechanically coupled to the drug delivery device. This known electronic module comprises a battery or a rechargeable accumulator as a power source.

It is an object of the present disclosure to provide improvements regarding recharging of a power source of an electronic module to be used with a drug delivery device.

This object is solved for example by the subject matter defined in claim 1. Advantageous embodiments and refinements are subject to the dependent claims. However, it should be noted that the disclosure is not restricted to the subject matter defined in the appended claims. Rather, the disclosure may comprise improvements in addition or as an alternative to the ones defined in the independent claims as will become apparent from the following description.

One aspect of the disclosure relates to an electronic encoder module for use with a drug delivery device. The module may be either connected to the drug delivery device for recording doses that are delivered from the drug delivery device or may be connected to a charging station for charging a power source of the module. In more detail, the electronic module may comprise a rechargeable electrical power supply, e.g. a rechargeable battery and/or an electric capacitor, a memory, a processor with a PCBA (printed circuit bard assembly) configured to control operation of the electronic module and coupled to the electrical power supply and to the memory, at least one sensor arrangement which is in communication with the processor, an, e.g. cup-shaped, coupling portion for releasably mounting the electronic module to the drug delivery device when it is not mounted to a charging station or to the charging station when it is not mounted to the drug delivery device, and at least one electrical power transfer component located within the coupling portion and connected to the PCBA and/or to the rechargeable electrical power supply. In other words, the module has to be detached from the drug delivery device for charging the rechargeable electrical power supply of the module by means of the charging station and, vice versa, the the module has to be detached from the charging station for recording doses that are delivered from the drug delivery device. Preferably, the coupling portion of the module is an interface for connecting the module with either the charging station or the drug delivery device.

The at least one electrical power transfer component may be adapted to supply electric energy to rechargeable electrical power supply by the charging station by means of direct conductive transfer when the coupling portion is mounted to the charging station. In this example, the charging station may comprise electrical contact pins or probes. The PCBA may comprise at least two contact pads facing into the cup-shaped coupling portion to be connected by pins of the charging station.

As an alternative, the at least one electrical power transfer component may be adapted to supply electric energy to rechargeable electrical power supply by the charging station by means of wireless energy transfer when the coupling portion is mounted to the charging station. In this example, the charging station may comprise a transmit charging coil. The electronic module may comprise at least one receive charging coil located facing towards the coupling portion and towards the coil of the charging station. Preferably, the coils are located in close vicinity when the module is attached to the charging station. In this alternative, the module may completely surround or encase the PCBA and the electronic hardware to provide a barrier against the ingress of water or particles. The PCBA may further comprise a receiver coil controller and the charging station may comprise a transmitter coil controller.

The drug delivery device and the charging station may use the same interface for releasable attachment with the module. For example, the coupling portion may be substantially cup-shaped, i.e. defining a cavity open on one side and suitable to receive an end of the drug delivery device, e.g. a dial grip, or a portion of the charging station, e.g. a socket comprising the contact pins or the coil. The coupling portion may further comprise a clip-on feature for axially and/or rotationally positioning the the electronic encoder module on the drug delivery device and/or on the charging station. In other words, the electronic encoder module may comprise attachment features for releasably mounting the module to the dial grip. For example, the attachment features may comprise at least one flexible clip for axially and rotationally fixing the button module to the dial grip. The at least one flexible clip may be received within a cap of the module and may extend substantially distally from a chassis of the module holding the sensor and the processor.

According to a further aspect of the present disclosure, the dial grip of the drug delivery device may comprise a mechanical coding and the electronic module may comprise a mechanical counter-coding which engages with the mechanical coding when the module is attached to the drug delivery device. This may prevent attaching a non-matching module to a drug delivery device. In addition, torque may be transmitted from the module to the dial grip via the mechanical coding and the mechanical counter-coding.

The sensor arrangement may be suitable for detecting a movement of an encoder of the drug delivery device which movement is indicative of doses that are dialed and/or delivered from the drug delivery device. In an example, the sensor arrangement is suitable for detecting a rotational movement of an encoder attached to a number sleeve of the drug delivery device indicative of doses that are delivered from the drug delivery device during injection. For example, the at least one sensor arrangement may comprise a first light source and a second light source as well as a corresponding first optical sensor and a corresponding second optical sensor. Preferably, when the module is mounted to the drug delivery device, the optical sensors are arranged to detect light emitted by the respective light source and reflected for example by the encoder of the drug delivery device. For example, the optical sensors may comprise a radiation detector and the light sources may comprise an electromagnetic radiation emitter, e.g. an LED, like an IR-LED, e.g. an NIR-LED.

In more detail, the electronic module may use a set of optical IR emitters/detectors to encode doses delivered from the drug delivery device, e.g. a variable dose pen. In order to reduce the level of stray IR radiation, for example from high ambient light levels if the pen is used outdoors, that may interfere with correct performance of the module sensors, the dial grip components of the pen may have features that are intended to reduce/suppress levels of reflected IR radiation. In particular, the button module components and/or the dial grip may contain an IR absorbing masterbatch and/or may have a spark-eroded textured surface finish.

The module may comprise an outer cap with a central axis, a chassis which is at least partially retained within the cap and a PCB or the PCBA comprising the memory and the processor. For example, the PCBA and the rechargeable electrical power supply may be retained in the cap and the chassis. Further, the light sources and the optical sensors may be arranged on a circular region about the central axis, with the first light source and the first optical sensor being angularly offset from the second light source and the second optical sensor. In addition, a light guide may be provided in the chassis guiding light from an internal light source to an external surface of the module in order to display information regarding the status or operation mode of the module.

In one example, the encoder and the optical sensor units are in a quadrature arrangement, i.e. they are a quarter wave out of phase, which means that if both light sources simultaneously emit light, only one sensor changes state for each unit dispensed. For example, this is achieved by providing two optical sensors circumferentially offset by n*30°+15° with n being an integer number. As the encoder and the sensor units are moved relative to each other, one of the optical sensors which previously received the light now does not receive the emitted light or vice versa. This may be achieved by the encoder selectively reflecting light. For example, a ring of teeth may be provided such that the teeth reflect light whereas the free space between adjacent teeth does not reflect light. As an alternative, areas reflecting light and areas absorbing light may be alternatedly provided. As a further alternative, the encoder may selectively block light. In other examples the encoder and the optical sensor units may be in anti-phase arrangement. In a still further alternative, the encoder and the optical sensor units are not in an anti-phase arrangement, such that if both light sources simultaneously emit light, none or only one or all optical sensors detect the light depending on the relative position of the encoder.

The processor of the electronic module may be configured to operate the sensor arrangement in a first low-power-consumption state and at least one further state having a higher power consumption compared with the first state. For example, the processor may be configured to regularly poll both optical sensors in the first low-power-consumption state, to maintain the first low-power-consumption state if the response of both optical sensors is identical to each other and identical to the preceding response, and to switch into the at least one further state if the responses of the optical sensors are different from each other or are different from the preceding response. When used for detecting the amount of dose injected from a drug delivery device, the processor may be configured to switch to the at least one further state upon detection of beginning of the respective dose delivery operation of the drug delivery device, and to switch to the first low-power-consumption state after the respective dose delivery operation has been finished. In an example, the module is, at least while the encoder module is mounted to the drug delivery device, permanently operated in the first low-power-consumption state, which may be a low accuracy mode, except while the measurement and recording system is operated in the at least one further state, which may be a high accuracy mode.

The electronic module may further comprise a communication unit for communicating with another device. Preferably, the electronic module is configured such that it may be switched from the first state having lower energy consumption into the second state having higher energy consumption, thereby inducing the communication unit to establish said communication with another device, e.g. a synchronisation or pairing operation. An electronic control unit may issue a command, e.g. a signal, to another unit of the electronic dose recording system such that this unit is switched on or rendered operational. This unit may be the communication unit for communicating with another device, e.g. a wireless communications interface for communicating with another device via a wireless network such as Wi-Fi or Bluetooth, or even an interface for a wired communications link, such as a socket for receiving a Universal Series Bus (USB), mini-USB or micro-USB connector. Preferably, the electronic module comprises an RF, WiFi and/or Bluetooth unit as the communication unit. The communication unit may be provided as a communication interface between the dose recording system or the drug delivery device and the exterior, such as other electronic devices, e.g. mobile phones, personal computers, laptops and so on. For example, dose data may be transmitted by the communication unit to the external device. The dose data may be used for a dose log or dose history established in the external device.

According to a still further aspect of the present disclosure, the electronic module further has a sleeping state in which the light sources are not activated (not provided with power from the power source). The electronic dose recording system may further comprise at least one switch and/or a motion sensor, e.g. an accelerometer, suitable for detecting movement of the electronic system. In this example, the processor may be configured to maintain the sleeping state if no switch activation or movement is detected by the at least one motion sensor and to switch into the first low-power-consumption state or into the at least one further state if a switch activation or a movement is detected by the at least one motion sensor. Generally, a sleeping state or mode may be a mode in which all functionalities of the module are at minimal or virtually zero power consumption but which does not require a system boot up in the event that the electronic system (or the drug delivery device) is taken out of sleeping mode.

The electronic module may further comprise a protection circuit for protecting the rechargeable electrical power supply from excessive current, excessive voltage and/or overcharging. In addition or as an alternative, the electronic module and/or the charging station may further comprise a battery charge control for controlling the charging of the rechargeable electrical power supply by the charging station.

A dose recording system according to an independent aspect of the present disclosure may comprise a drug delivery device as described above and an electronic encoder module for releasable attachment to the dial grip of the drug delivery device. For example, the module may comprise a sensor, a processor configured to control operation of the at least one sensor and to process and/or store signals from the at least one sensor, and a cap for receiving a proximal end of the dial grip. In more detail, a drug delivery device for setting and dispensing doses of a liquid drug may comprise a housing, a cartridge holder containing a cartridge filled with a liquid drug and a dose setting and drive mechanism with a dial grip and a dose setting drum. The dose setting and drive mechanism may be configured to perform a dose dialing operation for selecting a dose to be delivered by the drug delivery device by rotating the dial grip and the dose setting drum relative to the housing and a dose delivery operation for delivering the set dose by rotating the dose setting drum relative to the dial grip and the housing. Thus, if the dose setting drum comprises an array of encoder features, e.g. formed in a circular pattern, rotational movement of the dose setting drum relative to the dial grip may be detected by the module. Preferably, the dial grip may have at least one aperture that is coincident with at least a portion of the array of encoder features of the dose setting drum such that the sensors are able to detect the movement. When the module is releasably attached to the dial grip, the dial grip may be received in the, e.g. cup-shaped, coupling portion of the electronic module.

In the dose setting and drive mechanism a clutch may be provided acting between the dial grip and the dose setting drum which is engaged during the dose dialing operation so that the dial grip and dose setting drum rotate together and is disengaged during the dose delivery operation to allow relative rotational movement between the dose setting drum and the dial grip. The clutch may comprise a ring of clutch teeth provided at the distal end of the dose setting drum and a ring of corresponding clutch teeth provided on the dial grip. The complexity of the drug delivery device may be minimized if a component part has several different functions. According to an aspect of the present disclosure, the clutch teeth provided at the distal end of the dose setting drum may have the additional function of the encoder features. In other words, the relative rotational movement is detectable by a sensor of an electronic module to determine the size of the dose that is delivered. However, the present disclosure is not limited to the encoder features being the clutch teeth. Rather, the encoder features but could simply be flags for an optical encoder system.

The present disclosure further pertains to a drug delivery device with the electronic module as described above which drug delivery device comprises a cartridge containing a medicament.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders.

Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codeable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N-(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrome.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

An example drug delivery device may involve a needle-based injection system as described in Table 1 of section 5.2 of ISO 11608-1:2014(E). As described in ISO 11608-1:2014(E), needle-based injection systems may be broadly distinguished into multi-dose container systems and single-dose (with partial or full evacuation) container systems. The container may be a replaceable container or an integrated non-replaceable container.

As further described in ISO 11608-1:2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014(E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014(E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

The terms “axial”, “radial”, or “circumferential” as used herein may be used with respect to a main longitudinal axis of the device, the cartridge, the housing or the cartridge holder, e.g. the axis which extends through the proximal and distal ends of the cartridge, the cartridge holder or the drug delivery device.

A non-limiting, exemplary embodiment of the disclosure will now be described with reference to the accompanying drawings, in which:

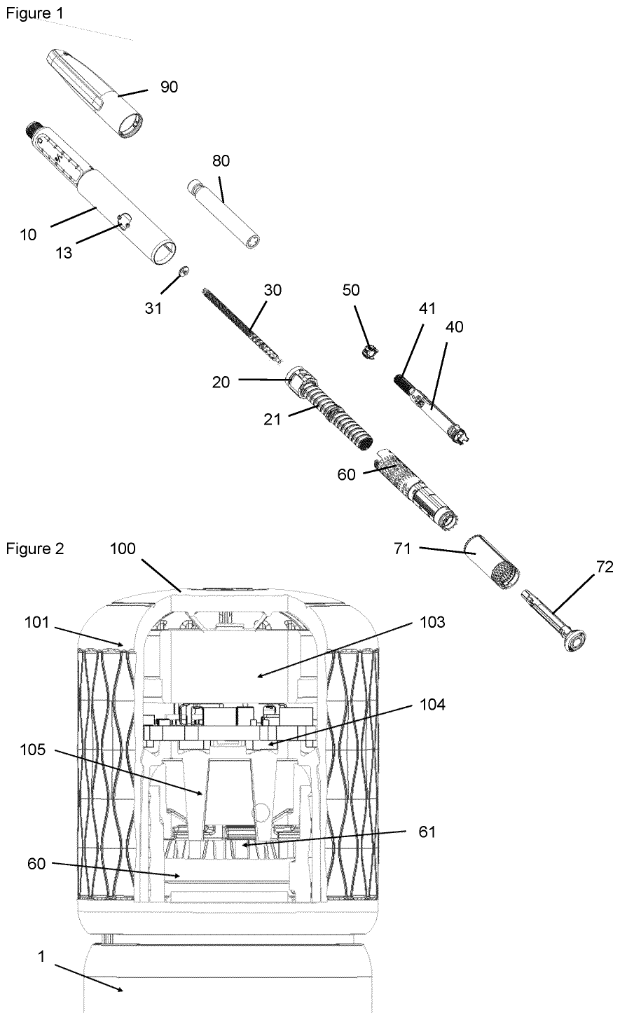

FIG. 1 shows the component parts of an embodiment of a drug delivery device;

FIG. 2 shows a partially cut away view of an end of the device of FIG. 1 together with an electronic encoder module;

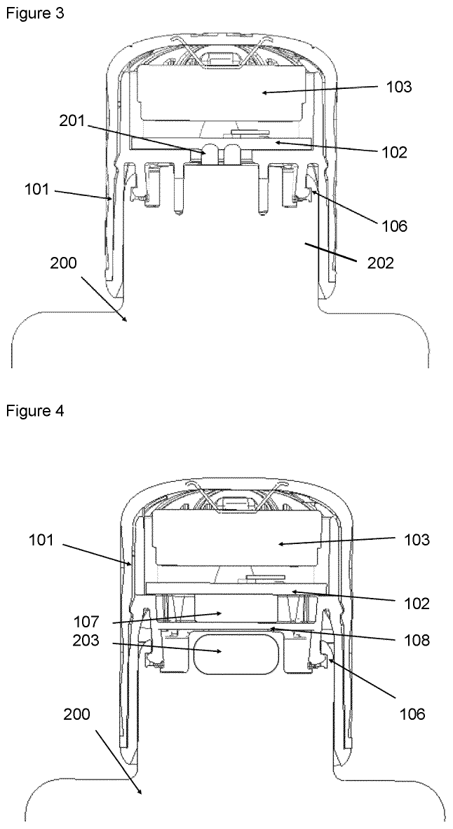

FIG. 3 shows a sectional view of the electronic encoder module according to an embodiment attached to a charging station; and

FIG. 4 shows a sectional view of the electronic encoder module according to a further embodiment attached to a charging station.

In the figures, identical elements, identically acting elements or elements of the same kind may be provided with the same reference numerals.

In the following, some embodiments will be described with reference to an insulin injection device. The present disclosure is however not limited to such application and may equally well be deployed with injection devices that are configured to eject other medicaments or drug delivery devices in general, preferably pen-type devices and/or injection devices.

Embodiments are provided in relation to injection devices, in particular to variable dose injection devices, which record and/or track data on doses delivered thereby. These data may include the size of the selected dose and/or the size of the actually delivered dose, the time and date of administration, the duration of the administration and the like. Features described herein include the arrangement of sensing elements and power management techniques (e.g. to facilitate small batteries and/or to enable efficient power usage).

The drug delivery device 1 shown in the FIGURES is based on the same general working principle as the disposable injection pen disclosed in EP 2 890 434 B1 to which reference is made regarding the main functions and the operation modes. However, the embodiment depicted in the Figures is improved in some aspects as described below in more detail and is adapted for use with a detachable electronic encoder module 100. Non-limiting examples of such a module are disclosed in WO 2021/116387 A1 and PCT/EP2021/060631 to which reference is made regarding the main functions and the operation modes of an electronic module.

Although described with reference to a disposable injection pen similar to the one disclosed in EP 2 890 434 B1, the present disclosure is applicable to other disposable or reusable drug delivery device, including but not limited to the injection devices disclosed in one of WO 2004/078239 A1, WO 2014/033195 A1, WO 2009/132777 A1, WO2005/018721, U.S. Pat. Nos. 5,693,027, 6,663,602 or 7,241,278.

“Distal” is used herein to specify directions, ends or surfaces which are arranged or are to be arranged to face or point towards a dispensing end of the drug delivery device or components thereof and/or point away from, are to be arranged to face away from or face away from the proximal end. On the other hand, “proximal” is used to specify directions, ends or surfaces which are arranged or are to be arranged to face away from or point away from the dispensing end and/or from the distal end of the drug delivery device or components thereof. The distal end may be the end closest to the dispensing and/or furthest away from the proximal end and the proximal end may be the end furthest away from the dispensing end. A proximal surface may face away from the distal end and/or towards the proximal end. A distal surface may face towards the distal end and/or away from the proximal end. The dispensing end may be the needle end where a needle unit is or is to be mounted to the device, for example.

FIG. 1 depicts the medicament delivery device or drug delivery device 1 comprising an outer housing 10, an inner housing insert 20, a piston rod 30, a drive sleeve 40, a nut 50, a dose setting drum 60, a dose setting member 70, a cartridge 80 and a cap 90. A needle arrangement (not shown) comprising a needle hub and e.g. a needle cover may be provided as additional components.

The outer housing 10 is a generally tubular element having a distal part, which forms a cartridge holder 11 for receiving cartridge 80 and which is provided with a thread 12 or the like for attaching a needle hub, and a proximal part, which encases component parts of a dose setting and drive mechanism. In a preferred embodiment, the outer housing 10 is transparent, with the proximal part being optionally provided with an opaque layer. The housing 10 comprises a transparent window 13.

The inner housing insert 20 is an inner body with a generally tubular configuration having different diameter regions. The inner housing insert 20 is received in the proximal part of housing 10 and is permanently fixed therein to prevent any relative movement of the inner housing insert 20 with respect to the housing 10. An external thread 21 is provided on the outer surface of the inner housing insert 20. Further, splines are provided on the inner surface of the inner housing insert 20 and its distal end is provided with an inner thread.

The piston rod 30 is an elongate element having two external threads with different leads, preferably of opposite hand which overlap each other. One of these threads engages the inner thread of the inner housing insert 20. A disk-like bearing 31 may be attached at the distal end of the piston rod 30 to allow relative rotation between the bearing 31 and the piston rod 30. As an alternative the bearing may be attached to the piston rod 30 as a one-piece component via a predetermined breaking point.

The drive sleeve 40 is a generally tubular element having different diameter regions. A distal region of the drive sleeve 40 has an external thread 41. An inner surface of the drive sleeve 40 has an inner thread engaging one of the external threads of the piston rod 30. The drive sleeve 40 surrounds the piston rod 30 and is at least partly located within inner housing insert 20. The drive sleeve 40 further has a flexible clicker arm formed by a U-shaped opening in the skirt of the drive sleeve 40. The clicker arm engages the internal splines in the inner housing insert 20 and is allowed to flex radially inwards during relative rotation between the drive sleeve 40 and the inner housing insert 20. This radial deflection is permitted as long as the clicker arm is in alignment with a pocket in the dose setting member 70, but may be blocked if the clicker arm is not in alignment with a pocket in the dose setting member 70, thereby preventing relative rotation between the drive sleeve 40 and the inner housing insert 20. At its proximal end, the drive sleeve 40 may comprise a spring arm abutting the dose setting member 70 and biasing the drive sleeve 40 distally with respect to the dose setting member 70. The internal surface of the drive sleeve 40 is provided with at least one axially extending groove permanently engaging splines on the dose setting member 70 such that any rotation of the dose setting member 70 is transferred to the drive sleeve 40.

The nut 50 is provided between the inner housing insert 20 and the drive sleeve 40. External ribs of the nut 50 engage the internal splines of the inner housing insert 20. An internal thread of the nut 50 engages the external thread 41 of the drive sleeve 40. As an alternative, splines and ribs could be provided on the interface between the nut 50 and the drive sleeve 40 and threads could be provided on the interface between the nut 50 and the inner housing insert 20. As a further alternative, the nut 50 may be designed as e.g. a half nut. Further, at least one, e.g. four, rotational hard stops are provided on nut 50 for interaction with corresponding stops on the drive sleeve 40 at the proximal end of thread 41.

The dose setting drum 60 is a generally tubular element with an internal thread engaging the external thread 21 of the inner housing insert 20. Thus, the dose setting drum 60 is interposed between the inner housing insert 20 and the housing 10. A series of numbers is provided, e.g. printed, on the outer surface of the dose setting drum 60 which forms a display member. The numbers are arranged on a helical line such that only one number or only a few numbers are visible in through window 13 of the housing 10. As will be explained in more detail below, the dose setting drum 60 is provided with a ring of clutch teeth 61 near its proximal end for engaging corresponding clutch teeth of the dose setting member 70. Further, an optional clicker arm may be provided, e.g. near the distal end of dose setting drum 60, for engaging a ratchet profile of the dose setting member 70.

The dose setting member 70 comprises two separate components, namely a tubular sleeve 71 and a button 72, permanently fixed to each other to function as a single component. The tubular sleeve 71 of dose setting member 70 comprises an internal ring of clutch teeth near its proximal end, an internal ring of ramped teeth forming the ratchet profile and stop teeth at its distal end for engaging counterstop teeth of the housing 10. The button 72 comprises a proximal end face and a stem extending distally from this end face. Two radial apertures and two axial apertures both formed in an outer groove which is concentrically disposed with respect to an inner groove formed in the end face of button 72 of dose setting member 70. The stem of button 72 is provided with the pocket into which the clicker arm of the drive sleeve 40 may flex and comprises at least one axially extending spline permanently engaging a respective groove formed in the drive sleeve 40.

As shown in FIG. 1, the tubular sleeve 71 of dose setting member 70 may be provided with a profiled proximal region facilitating gripping and rotating the dose setting member 70. The maximum external diameter of the dose setting member 70, especially of the tubular sleeve 71, is smaller than the external diameter of the housing 10. In more detail, the maximum external diameter of the dose setting member 70 is smaller than the internal diameter of a region of the housing 10 located adjacent to the dose setting member 70.

The cartridge 80 includes a pre-filled, necked-down cartridge reservoir, which may be typically made of glass. A rubber type bung or stopper is located at the proximal end of the cartridge reservoir, and a pierceable rubber seal is located at the other, distal, end. A crimped annular metal band is used to hold the rubber seal in place. The cartridge 80 is provided within the cartridge holder 11 with bearing 31 of piston rod 30 abutting the bung.

The cap 90 may be attached to the distal end of the device 1, thus covering the cartridge holder 11. The cap 90 may be releasable snapped onto the housing 10 and can be taken off for use of the device 1.

FIG. 2 shows the electronic encoder module 100 be releasably attached to the dial grip 70 of the drug delivery device 1, thereby forming a dose recording system. The module 100 may comprise a cup-shaped outer cap 101 retaining a PCBA 102 with a processor and a rechargeable power source 103. The module 100 further comprises a sensor arrangement 104 connected to the processor and operable to generate measurement data indicative of the dose setting operation and/or the dose delivery operation. For this purpose the sensor arrangement comprises at least one LED and one or more photo detector(s) together forming an optical sensor. Alternative sensor types could be implemented in addition to the LED and the photo detector or as an alternative thereto. Such alternative sensor types may include but are not limited to optical sensors, acoustic sensors, capacitive sensors, electrical switches. The PCBA 102 may further comprise or may be connected to a communication unit comprising e.g. a wireless Bluetooth® communication interface connected to the processor and operable to establish communication with another (external) device, e.g. a smartphone. The communication unit is operable to transfer data, e.g. measurement data, to said other device. Still further, an electronic user feedback generator may be connected to the processor and operable to generate a feedback signal to a user. In an exemplary arrangement, the electronic user feedback generator comprises an LED for generating optical feedback signals. In addition to the LED or as an alternative the electronic user feedback generator may comprise a sounder and/or a vibration motor.

The module 100 further comprises a chassis which is in snap-fit engagement with the cap 101, thereby axially and rotationally constraining the cap and the chassis. The chassis is transparent or translucent and may be made from Polycarbonate or the like. The chassis receives the PCB unit. Further, the chassis comprises two light pipes 105 which are axially extending protrusions of the chassis with polished side walls and diffuse end faces forming a sensor side end and an encoder side end. In other words, light may enter or leave the light pipes at the opposite sensor side end and encoder side end but is guided (reflected) by the side walls. In an example the light pipes 105 may be rotationally offset by 45° with respect to each other. The light pipes 105 are arranged to fit into or extend through one of the apertures in button 72 when the module 100 is attached to the drug delivery device 1. For this purpose, the cap 101 and the chassis form a coupling portion which has a cup shape in the depicted example, opening to the lower end of the module 100 in the Figures. In other words, the coupling portion formed by the cap 101 and the chassis define a void space of substantially cylindrical or conical configuration suitable for receiving the dial grip 70 of the drug delivery device or a socket of a charging station. In this coupling portion, the chassis further comprises attachment features in the form of two flexible clips 106 (see FIGS. 3 and 4) rotationally offset by e.g. 180° for engaging radial apertures in button 72.

The dose setting and drive mechanism of the drug delivery device is is configured to perform a dose dialing operation for selecting a dose to be delivered by the drug delivery device and a dose delivery operation for delivering the set dose.

During the dose dialing operation, clutch teeth 61 of the dose setting drum 60 engage clutch teeth of the dial grip 70. Thus, the dose setting drum 60, the dial grip 70 and the drive sleeve 40 (which is splined to the dial grip 70) are rotationally coupled. A user may set a dose by rotating tubular sleeve 71 of dial grip 70 such that the dose setting drum 60, the dial grip 70 and the drive sleeve 40 wind out of housing 10 along a helical path defined by thread 21 of the inner housing insert 20 guiding the thread of dose setting drum 60. The pitch of the threaded interface between the piston rod 30 and the drive sleeve 40 corresponds to the pitch of the threaded interface between the inner housing insert 20 and the dose setting drum 60 such that the piston rod 30 remains stationary held by its threaded interface with the inner housing insert 20. The selected dose amount is visible through window 13 in the housing 10. During this rotation of the dose setting drum 60, the dial grip 70 and the drive sleeve 40 relative to the housing, the flexible clicker arm of drive sleeve 40 snaps over the internal axial splines of the inner housing insert 20 thereby defining discrete rotational positions. In the depicted exemplary embodiment, one full rotation of the dial grip corresponds to 24 dose increments. For example, where the injection device 1 is configured to administer human insulin, the dosage increments may be displayed in so-called International Units (IU), wherein one IU is the biological equivalent of about 45.5 micrograms of pure crystalline insulin (1/22 mg).

At the maximum settable dose of, e.g. 80, units the stop features may engage to prevent further dialing. Further, during dose setting, the drive sleeve 40 rotates relative to the inner housing insert 20 such that the nut 50 which is rotationally fixed to the inner housing insert 20 via axially extending ribs and splines is moved on thread 41 of the drive sleeve 40. The last dose nut 50 provides the function of counting the number of dispensed units. The nut 50 locks the device 1 at the end of life and as such no more drug can be dialed or dispensed by the user. Rotation of the drive sleeve 40 during dialing causes the nut 50 to advance along thread 41. The nut 50 is free to slide axially within the inner housing insert 20 at all times which allows advancement of the nut 50. At the end of life condition, the stop features of the last dose nut 50 contact the corresponding features on the drive sleeve 40. The splined contact with inner housing insert 20 reacts any torque transmitted by these stop features 47.

With the desired dose dialed, the device 1 is ready for dose dispensing. This basically requires pushing dial grip 70 which will result in a disengagement of the clutch teeth 61 from the clutch teeth of the dial grip. As mentioned above, when dialing a dose the dial grip 70 is ‘biased out’ by the flexible arm of drive sleeve 40 and the clutch teeth which rotationally lock the drive sleev 40, dial grip 70 and dose setting drum 60 together are engaged. Upon pressing the dial grip 70 the clutch teeth disengage and relative rotation between the dose setting drum 60 and the dial grip 70 is possible. In all conditions the drive sleeve 40 and the dial grip 70 remain rotationally locked. Thus, with the clutch disengaged (dial grip 70 pushed in) dial grip 70 and drive sleeve 40 are rotationally locked together while the dial grip 70, the drive sleeve 40 and the dose setting drum 60 are still axially coupled. At the same time the relative axial movement of the dial grip 70 with respect to the drive sleeve 40 results in the pocket on the stem being shifted relative to the flexible clicker arm. Thus, the flexible clicker arm is prevented from flexing inwards. This activation of the lockout feature prevents the flexible clicker arm from overcoming splines of the inner housing insert 20 if the dial grip 70 is pressed. In this condition, the drive sleeve 40 and the dial grip 70 are rotationally constrained to the inner housing insert 20 thus preventing any rotation relative to the housing 10.

With the desired dose dialed the dial grip 70 can be depressed and the piston rod 30 is driven forward to dispense drug from the cartridge. The interaction of mating threads between the piston rod 30, drive sleeve 40 and inner housing insert 20 delivers a mechanical advantage of e.g. 2:1. During dose dispensing a dispense clicker is active which involves the ratchet profile of the dial grip 70 and the clicker arm of dose setting drum 60. The dispense clicker provides primarily audible feedback to the user that drug is being dispensed. Relative rotation is only allowed in one direction. During dose delivery, the drive sleeve 40 does not rotate relative to the inner housing insert 20. Thus, the rotational position of nut 50 is not changed during dose delivery and the nut 50 remains at its position on the drive sleeve 40 set during dose dialling.

The dial grip 70, more specifically the tubular sleeve 71, has a set of axially directed stop teeth at its distal end. These teeth are formed on the proximal rim of the tubular sleeve 71 and are configured to engage a set of counterstop teeth formed on an internal surface of the housing 10 at the end of delivery of a dose. These teeth control and ‘true-up’ the rotational position of the dial grip 70 with respect to the housing 10 and the dose setting drum 60 at the end of each dose. This is important to ensure that the clutch teeth between the dial grip 70 and the dose setting drum 60 are aligned so that they reengage correctly when the dial grip 70 is released (allowing a subsequent dose to be dialled). Furthermore, it is important for accurate function of the module 100 that the rotational position of the dial grip 70 is consistent with respect to the dose setting drum 60 at the end of each dose. This is because the module 100 is intended to accurately detect the total relative rotational movement of the clutch teeth 61 on the dose setting drum 60 relative to the dial grip 70 to determine the size of the delivered dose. Without the above mentioned teeth between the dial grip 70 and housing 10, the relative rotational position of these two parts at the end of a dose would only be determined by the dial clicker (flexible clicker arm of drive sleeve 40 and axial splines of inner housing insert 20). However, as the dial clicker is deliberately designed to be overcome with relatively low torque, it does not provide a particularly accurate rotational datum. Still further, if the dial grip 70 is pushed in with the pen 1 in the 0U dial condition the engagement of these teeth prevent the user from rotating the dial grip 70 relative to the housing 10. Rotating the dial grip 70 in this condition would allow the user to manipulate the pen mechanism in a way that might advance or retract the piston rod 30, potentially leading to dose errors.

The drug delivery device 1 is suitable to be used with or without module 100. If modue 100 is fited over dial grip 70, flexible clips 106 engage and snap into radial apertures of button 72, while light pipes 105 extend into axial apertures. When used in conjunction with module 100, the ring of clutch teeth 61 of the dose setting drum 60 has the additional function of an encoder.

These radially outwardly projecting clutch teeth 61 located on the outer diameter of the dose setting drum 60 may be detected by the optical sensor. In the exemplary embodiment, there are only twelve equi-spaced clutch teeth 61, yet they combine with a set of twenty-four mating teeth on the dial grip 70 to provide twenty-four discrete engagement locations (thus allowing 24 selectable dose increments per revolution of the dial grip when dialling and delivering doses). This arrangement has a number of advantages. Because the clutch teeth 61 project radially from the external diameter of the dose setting drum 60, they interact with the teeth formed on the inner diameter of the dial grip 70 to transfer torque at the largest possible diameter. This means that they are stronger and more resistant to torque applied by the the user. A smaller number of wide teeth (i.e. 12 ‘double width’ teeth rather 24 single width teeth) also provides a stronger means of transferring torque from the dose setting drum 60 to the dial grip 70. A smaller number of wide teeth 61 also provides a much better target for the optical detector of the module 2 because the individual reflective/non-reflective regions are wider and therefore provide better optical signal response and are more accommodating of angular tolerance variability (relative over or under travel) when the dose setting drum 60 rotates relative to the dial grip 70.

The dose button 72 of the pen injector 1 has two concentric grooves formed in its distal end surface. These grooves accommodate features of the module 100, allowing the module to be rotated relative to the dose button 72 to find the correct rotational alignment during attachment. The grooves serve as a means by which a set of features are discretely provided on the dose button 72 of the pen that prevent attachment of an incorrect type of button module 100, i.e. dedication features, allow the modue 100 to be securely attached via clip features 106 and provide access to clutch teeth 61 of the pen mechanism that allow the dose value to be detected/encoded by the module 100. As mentioned above, the features that are used by the module 100 to detect the size of the delivered dose are the clutch teeth 61 that are formed as an equi-spaced array of encoder features around the outer circumference at the distal end of the dose setting drum 60. Although, these encoder features may not need to be clutch teeth 61, but could simply be ‘flags’ for the optical encoder system rotating together with the dose setting drum 60, the arrangement of apertures is useful for permitting access to such encoder features if a module 100 is fitted onto pen 1.

As the module 100 uses a set of optical IR emitters/detectors to encode doses delivered from the pen 1, it is preferred to reduce the level of ‘stray’ IR radiation for example from high ambient light levels if the pen 1 is used outdoors that may interfere with correct performance of the module sensors. The dial grip 70 components may have features that are intended to reduce/suppress levels of reflected IR radiation. In particular, the tubular sleeve 71 and/or the button 72 may contain an IR absorbing masterbatch and/or may have a spark-eroded textured surface finish.

The electrical power required by the module 100 for its encoding system is supplied by the rechargeable electrical power supply 103, e.g. a rechargeable battery, within the module housing or cap 101. This rechargeable electrical power supply 103 can be recharged when necessary, by connecting the module 100 to a separate charging station 200, with its own power supply (e.g. mains supply). The module 100 can therefore be used independently of the charging station 200 for a number of days/weeks before it needs to be recharged.

Recharging could be carried out via direct conductive transfer, e.g. by pins 201 on a socket 202 of the charging station 200 contacting pads in the module. In FIG. 3 pins 201 contact corresponding pads on the PCBA 102. As an alternative, recharging could be carried out via wireless power transfer, e.g. inductive power transfer using respective coils 107, 203 as indicated in FIG. 4.

The provision of external power to the module 100 during charging may enable Bluetooth over-the-air firmware updates while the module 100 is externally powered.

The key benefit of these embodiments is that it enables the lifetime of the module 100 to be increased, since the lifetime of a similar device powered by a single-use battery is typically limited by battery capacity. This may provide an opportunity to reduce the effective cost of the device (i.e. average cost per use), as well as the effective environmental impact. Further, this also allows for a simplification of some mechanical aspects of the device. For example, the availability of power may mean that the sensors 104 can be kept on at all times (at low frequency) between doses, and can therefore be used to wake the dose capture system instead of using a physical switch. Having no requirement for a physical switch allows for a simplification of the waterproofing means, as well as negating the need for a switch mechanism and associated hardware which would potentially be complex and at risk of damage from the user. One option if the sensors are to be used in this way is to have the sensors 104 turned off while the device is in the supply chain, and enabled when the device is charged for the first time.

The separate charging station 200 is less constrained by size/portability, which may enable additional functionality of the system. For example, it may contain means of storing additional information about the device health and the dose history that it is not possible to store on the device itself for long periods (e.g. due to storage limitations). It may also be possible to use the charging station 200 to check the performance of the module's optical system when it is mounted, as it could provide a consistent optical target for measurements.

One example of an embodiment of such a system is described below. In this embodiment, constant moderate-frequency polling (e.g. 200 Hz) of a pair of optical sensors 104 in the module 100 is used in a low-power mode to detect when the user has begun to dispense a dose using the injection device 1. This constant low-frequency polling is used in the low-power mode following first use when the module 100 is inactive, i.e. a dose is not being recorded. It may also be used while the module 100 is in a packaging, but before it is fitted to an injection device 1.

When the button 72 is first pressed, one of the two optical sensors 104 will be occluded by one of the encoder teeth 61 on the dose setting drum 60 and the other will not. As the first unit of drug is dispensed, the dose setting drum 60 will rotate such that the optical sensor 104 which was initially occluded ceases to be so, and the optical sensor which was not initially occluded becomes so. The moderate-frequency polling of the optical sensors 104 will allow this change of occlusion state to be detected, and the module 100 will wake and immediately record the dose being dispensed.

In addition to the constant moderate frequency polling described above, the module 100 in this embodiment is powered by the rechargeable battery 103 within its housing 101. The module 100 is designed to be used with a separate charging station 200, on which the module 100 can be mounted and which recharges the module battery 103 from a mains electrical supply. Electrical connection of the module 100 to the charging station 200 is achieved by having two exposed contact pads on the module PCBA 102, which connect to corresponding pins/probes 201 on the charging station 200.

The charging station 200 is designed to hold the module 100 in a fixed position such that the electrical contacts are made and the module 100 is retained while it is being recharged. This is achieved by using clip features 106 that interface with corresponding features on the module 100, these can be the same features that allow the module 100 to be attached/detached from an injection device 1. Features on the module 100 that are used to provide dedication means to the injection device 1 may also be used to align the module 100 to the charging station 200.

The electronic circuit used for charging the rechargeable battery 103 contains a battery protection circuit and a battery charge controller. These could both be located in the module 100, simplifying the charging station 200. Alternatively, they could be split between the module 100 and the charging dock 200, which reduces the space required in the module 100. If both are located in the module 100, both functions may be provided by a single integrated circuit.

In an alternative embodiment, the battery 103 may be charged wirelessly. This embodiment is the same as the above mentioned embodiment, with the following changes: Power transfer from the charging station 200 to the module 100 is carried out by inductive power transfer rather than direct conductive transfer. The electronic hardware necessary for inductive power transfer is included in the module 100, for example a wireless charging coil 107. Since power transfer is wireless, the housing of the module 100 can completely surround the PCBA 102 and electronic hardware to provide a barrier against ingress of water or particles. This is shown in FIG. 4 by a closed wall 108 of the chassis forming an upper end of the coupling portion. The charging station 200 has the electronic hardware necessary for inductive power transfer, instead of pins/probes. There is no need for features that define and maintain the relative rotational orientation of the module 100 and the charging station 200. For inductive power charging, the electronic architecture is adapted to include a coil 203 and a controller for both the transmit (charging station 200) and receive (module 100) nodes. For this architecture the protection circuit, battery charge control and receive coil controller in the module 100 could be individual integrated circuits, or combined in one or two integrated circuits.

For both embodiments, feedback may be provided to the user on the charging mode and status in a number of ways. This could be using LEDs with different flashing sequences or colours, or via the Bluetooth communications for display in an app. App feedback could potentially include a reminder to the user to charge the module 100 when the battery state of charge is detected to be low.

A further aspect of the present disclosure is a method of powering a clip-on electronic encoder module 100 designed for use in conjunction with an injection device, using optical sensors 104 with a rechargeable battery 103, which can be recharged via a separate charging station 200. The charging station 200 allows transfer of electrical power from the charging station 200 to the module 100, and may be connected to mains in order to provide the necessary electrical power. Power transfer may be achieved via direct conductive transfer (e.g. pins/probes 201 on the charging station 200, which interface with corresponding conductive pads in the module 100) or via wireless power transfer (e.g. inductive power transfer).

The module 100 may be configured to be operated to remain awake at all times, in one of two different modes: a ‘moderate frequency polling’ mode; and a ‘high frequency dose capture’ mode. For the majority of the time, the module 100 stays in its ‘moderate frequency polling’ mode, where the optical sensors 104 are regularly taking measurements to determine whether a dispense is starting. In this mode, if rotational movement of the mechanism (encoder teeth 61) of the injection device 1 is detected by the sensors 104, the module 100 will switch to the ‘high frequency dose capture’ mode, which enables sufficient samplerate for accurate encoding of the delivered dose size. Following the completion of the dose, the module 100 will return to the ‘moderate frequency polling’ mode after a set time period.

Reference Numerals

| | | | |
|---|---|---|---|
| 1 | drug delivery device (pen) | 71 | tubular sleeve |
| | | 72 | button |
| 10 | housing | | |
| 11 | cartridge holder | 80 | cartridge |
| 12 | thread | | |
| 13 | dosage window | 90 | cap |
| 20 | inner housing insert | 100 | electronic button module |
| 21 | thread | 101 | cap |
| | | 102 | PCBA (processor) |
| 30 | piston rod | 103 | rechargeable power supply |
| 31 | bearing | 104 | sensor |
| | | 105 | light pipe |
| 40 | drive sleeve | 106 | flexible clip |
| 41 | thread | 107 | coil |
| | | 108 | wall |
| 50 | nut | | |
| | | 200 | charging station |
| 60 | dose setting drum | 201 | pin |
| 61 | clutch teeth | 202 | socket |
| | | 203 | coil |
| 70 | dial grip | | |

1. An electronic encoder module for use with a drug delivery device (1) for recording doses that are delivered from the drug delivery device (1), the electronic module comprising:
a rechargeable electrical power supply (103)
a memory,
a processor with a PCBA (102) configured to control operation of the electronic module and coupled to the electrical power supply (103) and to the memory,
at least one sensor arrangement (104) which is in communication with the processor, wherein the sensor arrangement (104) is suitable for detecting a movement of an encoder (61) of the drug delivery device (1) which movement is indicative of doses that are dialed and/or delivered from the drug delivery device (1),
a coupling portion for releasably mounting the electronic module (100) to the drug delivery device (1) when it is not mounted to a charging station (200) or to the charging station (200) when it is not mounted to the drug delivery device (1), and
at least one electrical power transfer component (107) located within the coupling portion and connected to the PCBA (102) and/or to the rechargeable electrical power supply (103).

2. The electronic module according to claim 1, wherein the at least one electrical power transfer component is adapted to supply electric energy to rechargeable electrical power supply (103) by the charging station (200) by means of direct conductive transfer when the coupling portion is mounted to the charging station.

3. The electronic module according to claim 2, wherein the PCBA (102) comprises at least two contact pads facing into the coupling portion to be connected by pins (201) of the charging station (200).

4. The electronic module according to claim 1, wherein the at least one electrical power transfer component is adapted to supply electric energy to rechargeable electrical power supply (103) by the charging station (200) by means of wireless energy transfer when the coupling portion is mounted to the charging station.

5. The electronic module according to claim 4, comprising at least one receiver coil (107) located facing towards the coupling portion and a transmitter coil (203) of the charging station (200).

6. The electronic module according to claim 5, wherein the PCBA (103) comprises a receiver coil controller and the charging station comprises a transmitter coil controller.

7. The electronic module according to any one of the preceding claims, wherein the coupling portion comprises a clip-on feature (106) for axially and/or rotationally positioning the the electronic encoder module (100) on the drug delivery device (1) and/or on the charging station (200).

8. The electronic module according to any one of the preceding claims, wherein the at least one sensor arrangement (104) comprises a first light source, a second light source, a first optical sensor (104) and a second optical sensor, wherein, when the module is mounted to the drug delivery device (1), the optical sensors (104) are arranged to detect light emitted by the respective light source and reflected for example by the encoder (61) of the drug delivery device (1).

9. The electronic module according to any one of the preceding claims, wherein the processor is configured to operate the sensor arrangement (104) in a first low-power-consumption state and at least one further state having a higher power consumption compared with the first state.

10. The electronic module according to any one of claims 8 or 9, wherein the processor is configured to regularly poll both optical sensors (104) in the first low-power-consumption state, to maintain the first low-power-consumption state if the response of both optical sensors (104) is identical to each other and identical to the preceding response, and to switch into the at least one further state if the responses of the optical sensors (104) are different from each other or are different from the preceding response.

11. The electronic module according to claim 10, wherein the processor is configured to switch to the at least one further state upon detection of beginning of the respective dose delivery operation of the drug delivery device (1), and to switch to the first low-power-consumption state after the respective dose delivery operation has been finished.

12. The electronic module according to any one of the preceding claims, wherein the rechargeable electrical power supply (103) comprises a rechargeable battery and/or an electric capacitor.

13. The electronic module according to any one of the preceding claims, further comprising a protection circuit for protecting the rechargeable electrical power supply (103) from excessive current, excessive voltage and/or overcharging.

14. The electronic module according to any one of the preceding claims, further comprising a battery charge control for controlling the charging of the rechargeable electrical power supply (103) by the charging station (200).

15. A drug delivery device for setting and dispensing doses of a liquid drug, the device comprising a housing (10), a cartridge holder containing a cartridge (80) filled with a liquid drug and a dose setting and drive mechanism with a dial grip (70) and a dose setting drum (60), wherein the dose setting and drive mechanism is configured to perform a dose dialing operation for selecting a dose to be delivered by the drug delivery device by rotating the dial grip (70) and the dose setting drum (60) relative to the housing (10) and a dose delivery operation for delivering the set dose by rotating the dose setting drum (60) relative to the dial grip (70) and the housing (10), wherein the dose setting drum (60) comprises an array of encoder features (61) formed in a circular pattern at its distal end for detecting rotational movement of the dose setting drum (60) relative to the dial grip (70), wherein the dial grip (70) has at least one aperture that is coincident with at least a portion of the array of encoder features (61) of the dose setting drum (60), and wherein an electronic module (100) according to any one of the preceding claims is releasably attached to the dial grip (70) such that the dial grip (70) is received in the coupling portion of the electronic module (100).
